(19) United States
(12) Patent Application Publication
(10) Pub. No.: US 2010/0162488 A1
(43) Pub. Date: Jul. 1, 2010

(54) PATIENT HANDLING SYSTEM

(75) Inventors: Hans Dahlin, Uppsala (SE); Kjell Westerlund, Uppsala (SE)

Correspondence Address: KEVIN FARRELL;PIERCE ATWOOD, ONE NEW HAMPSHIRE AVENUE, PORTSMOUTH, NH, 03801, US

(73) Assignee: ONCOlog Medical QA AB, Uppsala (SE)

(21) Appl. No.: 12/607,944

(22) Filed: Oct. 28, 2009

Related U.S. Application Data
(60) Provisional application No. 61/109,346, filed on Oct. 29, 2008.

(51) Int. Cl. A61G 1/00; A61B 6/04

(52) U.S. Cl. 5/600; 378/209

(57) ABSTRACT

One embodiment of the present invention relates to a system for transporting a patient between a plurality of diagnostic and/or therapy work stations. At each work station, a diagnostic and/or therapy process can be performed on the patient while the patient is attached to a table plate. The patient remains continuously attached to the table plate during a first diagnostic and/or therapy process at a first diagnostic and/or therapy work station, during transport to a second diagnostic and/or therapy work stations and during a second diagnostic and/or therapy process at said second diagnostic and/or therapy work station.

### BACKGROUND

1. Technical Field of the Invention

The present invention relates to a system and a method for handling patients.

2. Discussion of Background Information

Radiation therapy is used to treat patients with tumours. A radiation treatment unit is very expensive and to be economically efficient it should have a very high degree of utilisation. The treatment itself is very quick and usually takes just a matter of minutes, but there are delays in the current systems for handling patents which result in long downtimes for the therapy unit. Typically a system for treating a patient comprises a preparation station where a patient is immobilized on a gurney. The patient is then transported, normally walking, from the preparation room and placed on a patient supporting surface at a diagnostic station where the position of the treatment volume (i.e. the volume of patient tissue which is to be treated) is determined with the patient immobilized in the same way as was performed in the preparation room.

A computerized treatment plan including radiation beam configuration is prepared based on information resulting from diagnostic unit. After the treatment plan preparation the patient is transported, normally walking, to a radiation therapy station. The patient is placed on a treatment table plate and immobilized in the same way as was performed in the preparation room. The radiation treatment according to the treatment plans is then performed. When the treatment is finished the patient descends from the table plate and normally leaves the treatment room by walking. The radiation procedure is repeated 25 to 35 times over approximately one month. During the treatment series, checkups at the diagnostic unit can be performed to verify the stability of the patient immobilization related to the treatment volume. The need for easy and quick patient handling is essential to maximize the number of patients who can to be treated on the radiation therapy unit in order to maximise cost efficiency. The specific geometry of radiation therapy gantries makes it difficult to use unmodified standard treatment tables as platforms for a patient handling system. In particular, a smooth patient flow between diagnostic units and therapy rooms is required to ensure correct position of the treatment volume during radiation treatment.

### SUMMARY OF THE INVENTION

The objective of the present invention is to overcome at least some of the drawbacks of the prior art. This is achieved by the system and method as defined in the independent claims.

One embodiment of the present invention relates to a system for transporting a patient between a plurality of diagnostic and/or therapy work stations. At each work station, a diagnostic and/or therapy process can be performed on the patient while the patient is attached to a table plate. The patient remains continuously attached to the table plate during a first diagnostic and/or therapy process at a first diagnostic and/or therapy work station, during transport to a second diagnostic and/or therapy work stations and during a second diagnostic and/or therapy process at said second diagnostic and/or therapy work station.

It is one object of the present invention to provide a system for transporting a patient between different diagnostic and therapy work stations such that the transport is smooth and as painless as possible for the patient.

It is another object of the present invention to provide a system for transporting a patient between different diagnostic and therapy work stations such that the organs within the patients body remain at rest and substantially fixed in their relative positions during the time between locating the target irradiation spot and irradiation of the target irradiation spot.

Further embodiments of the invention are defined in the dependent claims. Other objects, advantages and novel features of the invention will become apparent from the following detailed description of the invention when considered in conjunction with the accompanying drawings and claims.

### BRIEF DESCRIPTION OF THE DRAWINGS

Preferred embodiments of the invention will now be described with reference to the accompanying drawings, wherein:

FIG. 1a-b) illustrates schematically an embodiment of a patient table top in accordance with the present invention.

FIG. 2a-c) illustrates schematically an embodiment of a docking unit in accordance with the present invention.

FIG. 3a-b) illustrates schematically an embodiment in accordance with the present invention of a transport trolley with a patient table top.

FIG. 4a-b) illustrates schematically an embodiment of a patient loading station in accordance with the present invention.

FIG. 5 illustrates schematically an embodiment in accordance with the present invention of a system for radiation therapy.

### DETAILED DESCRIPTION

The invention relates to a patient handling system for radiation therapy, and a method for carrying out such a therapy. An embodiment of a complete system in accordance with the present invention comprises a plurality of components that facilitate the handling of a patient undergoing radiation therapy. The radiation therapy is preferably a proton radiation therapy.

Components comprised in this embodiment of a patient handling system in accordance with the present invention are patient table plate, docking units, transport trolleys and patient loading stations.

A patient table plate is an important component of the system. The patient table plate is preferably constructed so that it can be used in many types of diagnostic and therapy units. Preferably it is manufactured from non-magnetic and non-conductive materials in order to work well in MR systems, preferably it is radio translucent with very low radiation attenuation in order to work well with CT and x-ray units (simulators) and preferably it can have a homogeneous core material and low atomic number reinforcing fibres for minimal distortion of a radiation beam such as for example a proton beam. Preferably it has indexing features for reproducible repositioning of patient immobilization shells and preferably it also has adaptors for secure and precise connection to the docking units (described below) at the patient supporting tables at the diagnostic and therapy units.

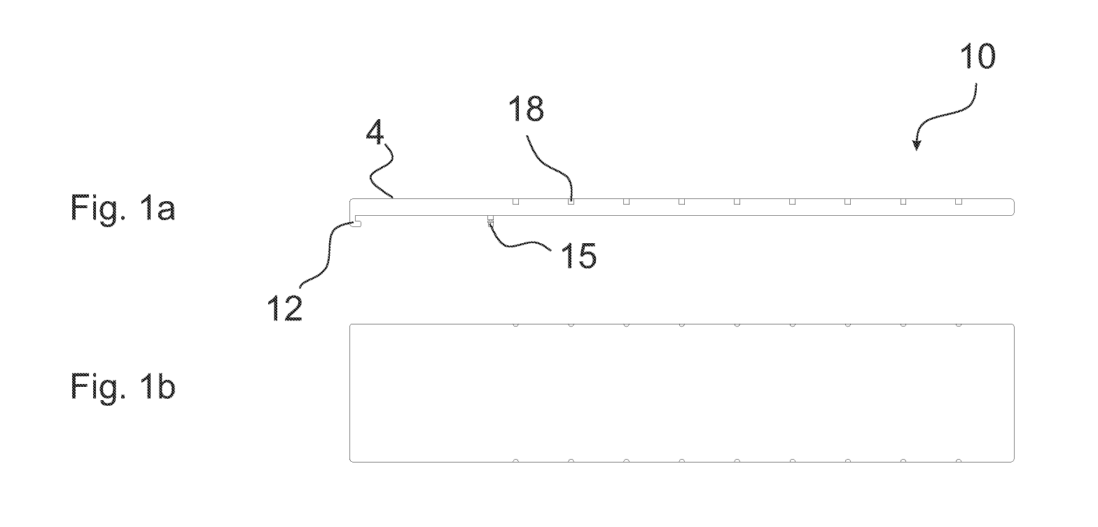

In one embodiment of the present invention shown in FIGS. 1a and 1b, the patient table plate 10 is an elongated board with a head end 4 and a base end 6, and it is suitable for supporting a patient in a laying down position. It is preferably manufactured from radiation-translucent, non-magnetic and non-conductive composite material in order to be compatible for radiation therapy such as for example CT, MR, X-ray and proton therapy. The external surface is preferably non-porous and hard for easy cleaning with conventional disinfecting agents. It has indexing features 18 which are adapted to a patient fixation shell system that is selected by the customer. A fixation shell is a form which conforms to the contours of a specific patient (i.e. it is a patient-specific immobilization shell) in order to make sure the patient stays in a fixed position, which is substantially the same position for every subsequent treatment occasion. This restricted movement of the patient helps ensure that the volume of the patient being scanned is as near as possible the same as the volume being treated. A patient fixation shell can be fitted to the patient table plate using the indexing features so that each time the patient is treated the patient can be positioned in exactly the same position on the table plate. The fixation shell is applied on top of the patient table plate 10, such that the unit comprising both the table plate and the fixation shell is custom-fitted and individually-adapted for each patient.

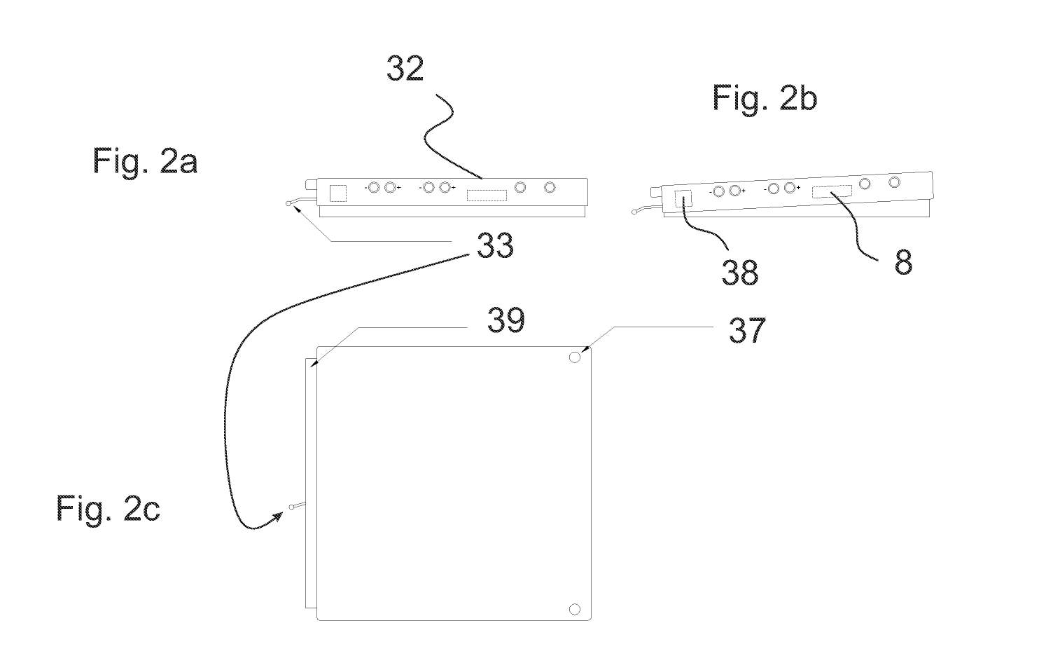

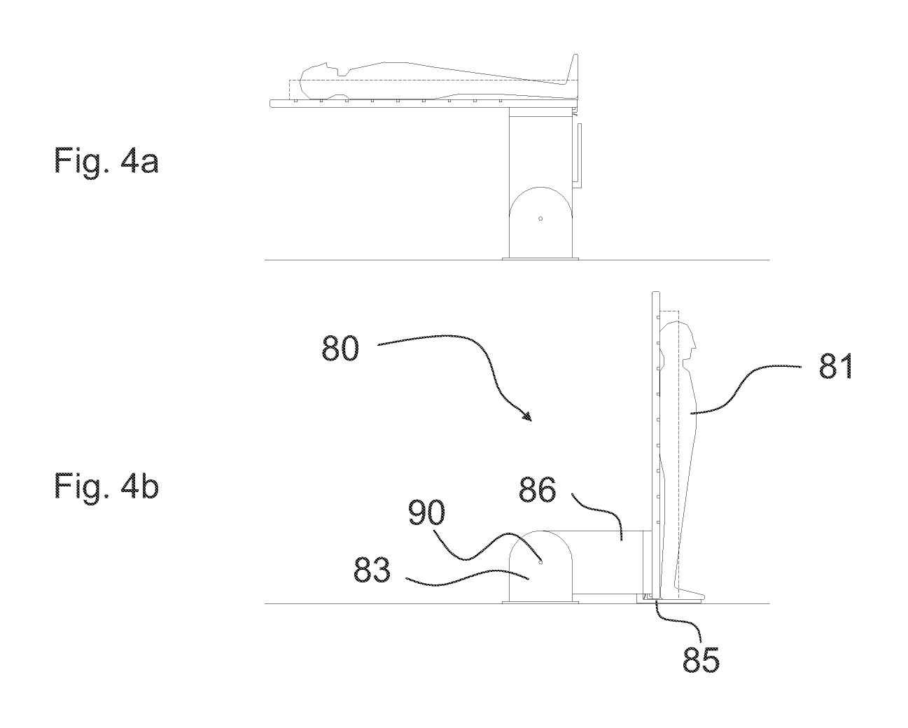

At the base end 6 the table plate has registration pins 15 for accurate repositioning of the table plate on docking units and locking parts 12 for securely connecting it to a docking unit 30. An example of a docking unit 30 can be seen in FIG. 2a-c. Docking unit 30 comprises a lock and release control 33, a locking mechanism 39 adapted to mate with the locking parts on the patient table plate 10, and a supporting surface 32 upon which the head end 4 of the patient table plate can be attached and supported substantially immovably in a cantilever manner. The docking unit is further provided with registration holes 37, adapted to receive the registration pins 15 of the patient table plate 10, to help ensure a precise and safe assembly. The table plate 10 may be provided with a data memory 8 for temporary storage of parameters for the automatic set-up of pitch and roll angles associated with the patient being treated. This memory can be accessed by a microprocessor 38 in the docking unit 30. Preferably the table plate is sufficiently stiff and rigid so that it is substantially not deformed during normal use and therefore can provide very precise patient positioning and reliable support during all the diagnostic, transport and therapy procedures. Preferably a patient remains in the same position on the same table plate 10 throughout the whole diagnostic and therapy procedure such that the patient and table plate can be considered to be as a single geometrically well-defined object. This is intended to enable precise and reproducible positioning of the patient in the diagnostic and therapy units. The patient remains on the table plate 10 from the loading at a loading station 80 (seen in FIG. 4a-b) through CT or MR localization, x-ray localization in the treatment room and proton treatment, up to unloading again in a loading station 80, and the table plate 10 and patient are treated as a single object throughout the process.

A docking unit 30 is mounted on each of the patient table bases associated with each particular diagnostic and therapy unit. A table base is a rigid support, usually mounted on the floor, which holds the docking station in a known and substantially immovable position. Each docking unit 30 provides an interface between the different existing table bases and the patient table plate 10. In one embodiment of the present invention, a docking unit 30 is permanently mounted on each table base and is adapted to receive the head end 4 of a patient table plate. Each docking unit has a locking mechanism 34 for connecting the patient table plate 10 quickly, securely and accurately to the table base. Preferably each docking unit also has a system for motorized fine adjustment of pitch and roll of a patient table plate 10 attached to it. Preferably, a motorized adjustment of tilt and roll of up to ±5°, more preferably up to ±3° in each direction is possible. This adjustment can be made manually with control buttons 36 on the docking unit 30. Alternatively a desired position can also be stored, either in the memory circuit in the patient table plate 10 or in an external database. The stored desired position values may then be used for automatic set-up of pitch and roll angles through the internal microprocessor controller of the docking unit. A preferably low voltage power supply and external data communication system for the docking unit 30 can be provided by conductors passing through the existing table base.

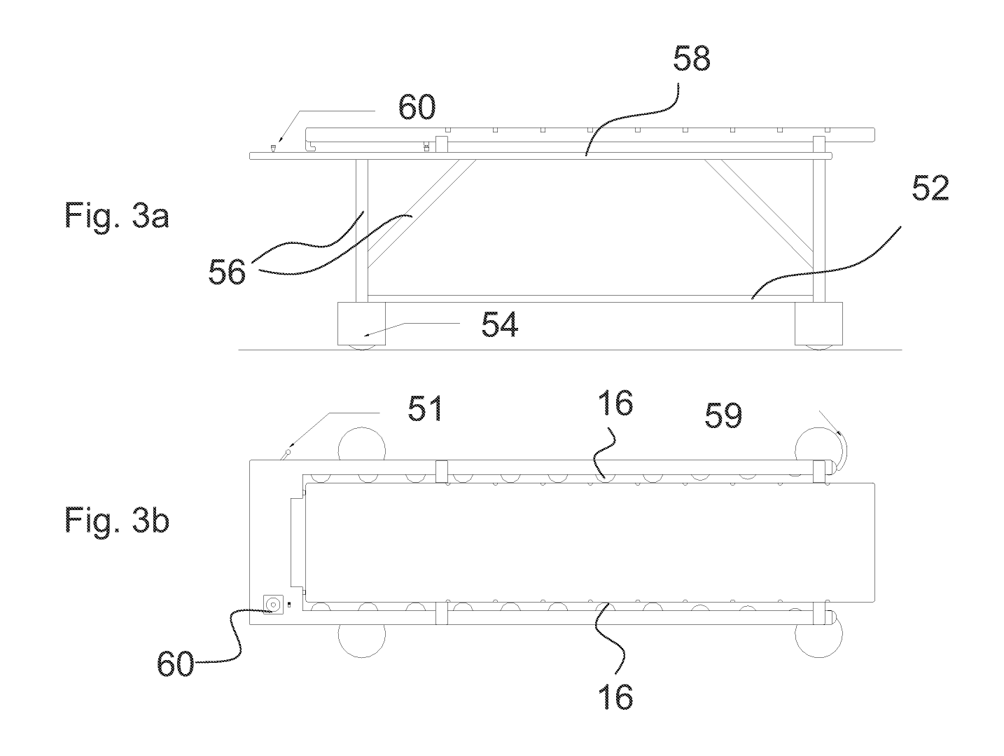

In one embodiment of the present invention, a transport trolley 50 as shown in FIG. 3a-b is provided. Transport trolley 50 preferably has a wheel base 52 with, for stability, preferably four wheels 54 and supporting legs 56 which support a nominally horizontal table 58 which is adapted to receive a patient table plate. Some or all of the wheels 54 are preferably motorized to facilitate movement of the trolley. The table 58 of the trolley further has a plurality of guiding rollers 16 that the patient table plate 10 can be guided by to make sure connection and disconnection of the patient table plate 10 is easy and smooth, and a release handle 51 for easy disconnection of the patient table plate 10. The transport trolley 50 is designed to hold the patient table plate 10 securely during transport between different stations, with or without the patient on it, and to allow easily connection and disconnection of patient table plates 10 at docking stations. Preferably it is wide enough to straddle the table bases for docking. The transport trolleys 50 facilitate the connection and disconnection of the patient table plates 10 to the docking units 30 and easy transport between the different stations.

Preferably transport trolleys 50 are motorized and have a steering system 60 to make the transport safe and easy between stations and through the narrow passages in the radiation exposure-limiting maze associated with some types of radiation therapy systems. In addition, the transport trolley can have an “autoguidance” system with sensors or digital cameras 59 that can follow a preinstalled path in the floor. This path may consist of a signal wire that is installed under the flooring or special tape attached to the floor which the trolley can follow.

The power supply to the transport trolley 50 may be an internal rechargeable battery that may be charged at night or other times when the trolley is not in use. The dimensions of the transport trolley 50 are preferably adapted so that they can pass without interference over existing treatment table bases.

The transport trolleys 50 with patient table plates 10 may also be used to transport fixation shells (not illustrated) to and from the storage area to avoid unnecessary repeated handling of them. A fixation shell is a form adapted for an individual patient, to make sure the patient stays in a fixed position, which is substantially the same position for every subsequent treatment occasion. The fixation shell is applied on top of the patient table plate 10, such that the unit comprising both the table plate and the fixation shell is individualized.

A separate patient loading station and unloading station 80 (called “loading station” from now on for brevity) can be used to speed up the patient loading process, which can be a major obstacle in the otherwise streamlined patient flow and it also provides much better reproducibility of the position of the internal organs of patients 81. The loading station 80 comprises two parts; a fixed part 83 which is not moving and is solidly mounted onto for example a floor, and a moving part 86 onto which the patient table plate 10 can be attached, that can be rotated around a rotation axis 90 through an angle of about 90° from a vertical position to a substantially horizontal position and vice versa. This allows a patient to be rotated from a vertical (standing or sitting) position to a horizontal (lying) position. It preferably has a footrest 82 that can be set to different distances from the base end in order to accommodate the needs of different patients, e.g. disabled patients or very short patients. If placed towards the foot end of the loading station 80 it acts as a foot rest 85 for standing patients who are being loaded onto a patient table plate 10. If positioned at a greater distance from the foot end of the loading station 80 it can act a seat for seated patients who are being loaded onto a patient table plate 10.

The time consuming, troublesome and sometimes painful procedure of getting old and/or sick patients into their fixation shell is greatly improved by the patient loading station 80. In one embodiment of the present invention, seen in FIG. 4a-b, the patient table plate 10 is docked to the loading station 80 such that the patient table plate 10 can be driven between a substantially vertical and a substantially horizontal position. The fixation shell can either be on the table plate 10 already (e.g. if it has been transport on the table plate from the storage room) or it can be connected to the table plate 10 in the vertical position (if it has been brought to the set-up room separately). If required, the shell can be positioned vertically on the loading station and patient table plate assembly, so that the patient can now just stand up on the foot rest 85 of the loading station 80, step into the fixation shell and turn until reference marks, such as skin marks applied on the body of the patient, agree with laser lines used as guidelines in the positioning of the patient, and the patient's body fits well in the shell. The foot rest 85 is adjustable in height to adapt to the needs of different patients. The loading station 80 is then lifted up, by a motor or manually, and turned into its horizontal position. The transport trolley 50 is brought forward and the table plate is undocked from the loading station to the trolley 50 for further transport to the next station. Unloading the patient is the same procedure done in reverse. This method saves time, provides much easier and dignified motions for the patient and gives excellent reproducibility of inner organ positions. The patient loading station 80 is attached to the floor in for example a patient set-up room and preferably it is powered by mains electricity.

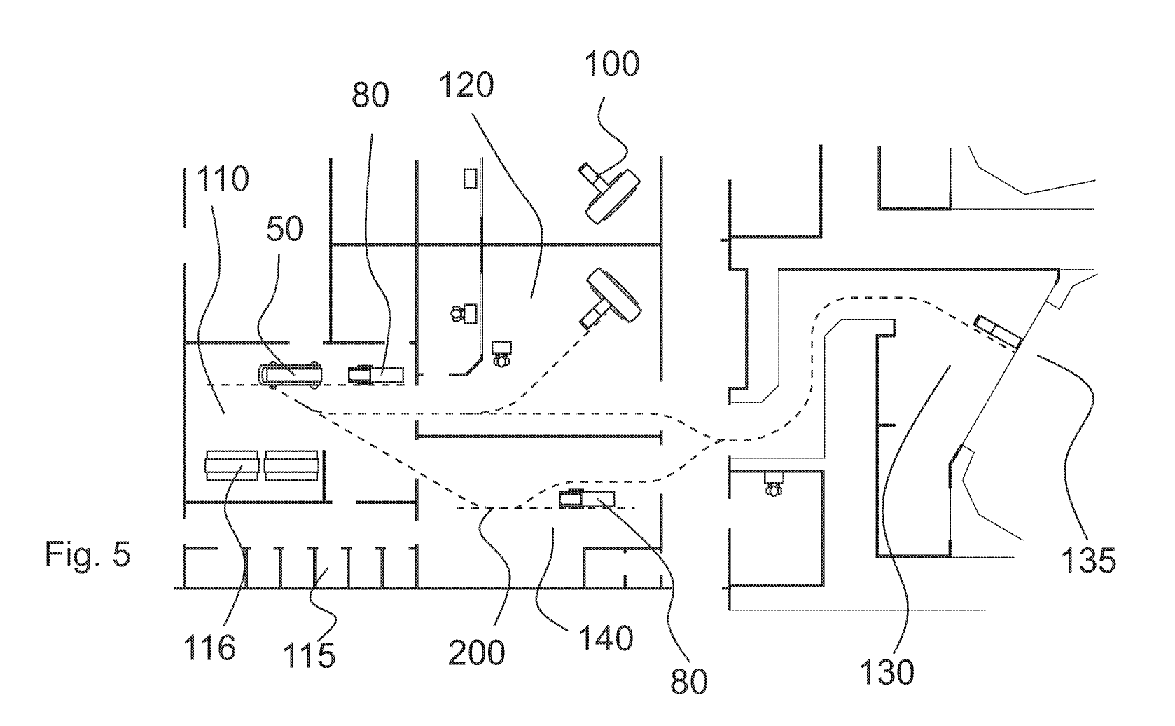

In one embodiment of the present invention as shown in FIG. 5, a system for transporting a patient between different stations for proton therapy is shown, the system comprising a minimum of a mounting room 110, a set-up room 120, a treatment room 130, a dismounting room 140, a patient loading station 80, dressing cabins 115, a patient unloading station 80 (loading and unloading can be one and the same station), a patient table plate 10, a docking unit 30, and a transport trolley 50 adapted to receive the patient table plate.

A patient is loaded onto the transport trolley 50 comprising a docking unit 30 and patient table plate 10, individualized with the fixation shell, in the mounting room 110. The transport trolley 50, now comprising also the patient, is then moved to the set-up room 120. In the set-up room, the patient is prepared for the radiation treatment, preferably a proton radiation treatment, by locating accurately, using for example CT, MR or x-ray, the location of the target area within the body of the patient. The transport trolley 50 is then moved to the treatment room 130 where the radiation treatment is performed. The transport trolley 50 can then be moved to the dismounting room 140 comprising a loading station 80, where the patient can be unloaded. The mounting room 110 and the dismounting room 140 can be the same room, with one loading station, but preferably the mounting room 110 and dismounting room 140 are separate rooms comprising one loading station 80 each, one being used for loading and one being used for unloading.

For the maximum throughput and utilization of the treatment resources it is desirable to have several sets of patient table plates 10 and associated transport trolleys 50 for each treatment room 130 as this allows the system to accommodate a plurality of patients at the same time, each at a different position in the system.

Such an arrangement permits simultaneous work by multiple therapist teams in several stations for example at a patient loading 80 and unloading stations 80, a simulation station, a CT station, an MR station, an x-ray station etc. By having several therapists working in parallel at several stations, efficient use of the proton radiation unit is possible.

In a preferred system, for maximum flexibility we suggest a minimum of three sets of patient table plates 10 and transport trolleys 50 for each one of the three treatment rooms 130.

Each station should preferably also be equipped with a docking unit 30 mounted on the existing table base supplied with each system (CT, MR, simulator and proton treatment unit).

Each room for loading and unloading a patient should preferably also have two patient loading stations 80, one for getting into the fixation shell and one for getting out.

Now, a method according to the present invention will be described, that utilises the patient handling system for proton therapy. The method allows for several patients to be active in the system simultaneously. The patient is kept on one table plate 10 during the entire treatment, and can easily be moved between different stations. The loading station 80 can recline the patient during loading and unloading, thereby providing a safe and dignified way for the patient to enter and exit the patient table plate 10 at the loading station 80. It should be kept in mind, that the patients going through this kind of treatment can be very weak and thus not strong enough to climb up and onto the patient table plate 10 or to a fixation shell at the patient table plate 10 by themselves.

In one embodiment of the present invention, the method comprises the steps of;
loading a patient in a fixation shell onto the patient table plate 10, mounted on a transport trolley 50, at a loading station 80;
moving the transport trolley 50 to a station where diagnostic imaging such as computerized tomography (CT) or magnetic resonance imaging (MR) can be performed;
performing diagnostic imaging e.g. CT or MR;
moving the patient to a station comprising a radiation therapy unit 135;
performing radiation therapy;
moving the patient to an unloading station 80, and unloading the patient.

The patient is loaded onto the patient table plate 10 described above. The loading station 80 can be tilted to a vertical position, thus making it possible for the patient to be loaded in an upright position, and placed in for example a fixation shell mounted on the patient table 10, the loading station 80 then reclines the patient into the horizontal position.

When the patient is in a horizontal position on the table plate 10 the transport trolley can be attached to the table plate 10 and can move it out from the loading station while the patient is secured in a fixed position within the fixation shell. Thus, the patient can be moved to for example a therapy room, where the patient table plate 10 can be disconnected from the transport trolley 50 and released onto a table base 100 on which the therapy can be performed, by raising the table base 100 when the patient table top 10 is in position above the table base 100, and then remove the transport trolley 50 which is ready for a potential new task. The patient table top 10 can in the same manner be connected to the transport trolley 50 again, by positioning the transport trolley under the patient table top 10 connected to the table base 100, lowering the table base such that the patient table top 10 rests on the transport trolley and is released from the table base 100, and then move the transport trolley 50 with the patient table top 10 from the table base 100. The transport trolley is preferably adapted so as to smoothly mate with the table base 100, releasing the patient table top 10 onto the table base 100, and then smoothly move away from the table base. Table bases are preferably provided in all therapy rooms, and adapted to firmly and safely hold the patient table top 10 during therapy until the patient table top 10 is connected to the transport trolley 50 again. The transport trolley 50 comprising the patient can then be moved to a station for CT or MR analysis. At this station the patient is scanned in preparation for the radiation therapy. By CT or MR analysis, a target area for the proton radiation is localised, and when the exact location has been determined in the body of the patient, it is important that the patient is fixed in position in the fixation shell.

After the target area has been localised, the transport trolley 50 comprising the patient is moved to the radiation therapy unit 135 where the radiation treatment is performed.

When the radiation treatment is ready, the transport trolley 50 comprising the patient is moved to the loading station 80 again, for unloading. After unloading, the transport trolley 50 can be equipped with a new individualized fixation shell for a new patient, for example stored in a fixation shell storage 116, and thus follow the same route again for the new patient.

In another embodiment of a method of the present invention, the transport trolley 50 is motorized. A motorized transport trolley 50 can follow a defined route 200 in the floor, for example the route can be defined by embedding magnets, a magnetic loop or a magnetic wire in the floor, or attaching tapes that can be recognized by a digital camera system. The signal from the magnetic means in the floor can be detected by the trolley 50, such that the trolley is guided along the route defined by the magnetic means. This enables a safe transport of the trolley 50 through the different stations, and makes the working environment for a person transporting the transport trolley 50 much more friendly, and decreases the risk for that person to end up having work related injuries. In addition the system with a motorized trolley 50 is helpful in providing smoother patient transportation and avoiding accidental bumps.

While the invention has been described in connection with what is presently considered to be the most practical and preferred embodiments, it is to be understood that the invention is not to be limited to the disclosed embodiments, on the contrary, is intended to cover various modifications and equivalent arrangements which are within the scope of the appended claims.

What is claimed is:

1. A system for transporting a patient between a plurality of diagnostic and/or therapy work stations where at the work stations a diagnostic and/or therapy process is performed on the patient wherein the patient is attached to a table plate and remains continuously attached to the table plate during a first diagnostic and/or therapy process at a first diagnostic and/or therapy work station, during transport to a second diagnostic and/or therapy work stations and during a second diagnostic and/or therapy process at said second diagnostic.

2. A system for transporting a patient according to claim 1, wherein at least one therapy work station comprises a proton radiation therapy unit.

3. A system for transporting a patient according to claim 1, wherein said system comprises at least one patient table plate and/or at least one docking unit and/or at least one transport trolley and/or at least one patient loading station.

4. A system for transporting a patient according to claim 1, wherein the at least one transport trolley comprises an autoguidance system, such that the transport trolley is able to sense a pre-installed guide path in the floor and hence automatically follow the guide path during transportation.

5. A system for transporting a patient according to 4, wherein the transport trolley is motorized.

6. A system for transporting a patient according to claim 1, wherein it further comprises a patient-specific fixation shell and the patient table plate comprises indexing features arranged such that a patient-specific fixation shell can be substantially identically positioned and repositioned at the patient table plate.

7. A system for transporting a patient according to claim 1, wherein the patient table plate is made of a radio-translucent, non-magnetic and non-conductive composite material.

8. A method for treating a patient comprising the steps of loading a patient onto a table plate, transporting said patient and table plate to a first diagnostic and/or therapy work station, performing a first diagnostic and/or therapy process at said first diagnostic workstation, transporting said patient and table plate to a second diagnostic and/or therapy work station, performing a second diagnostic and/or therapy process at said second diagnostic workstation while keeping said patient continuously attached to said table plate.
